(19) United States
(12) Patent Application Publication
(10) Pub. No.: US 2006/0058591 A1
(43) Pub. Date: Mar. 16, 2006

(54) First-response portable recorder and automated report generator

(75) Inventors: Dennis P. Garboski, Bedford, NH (US); Thomas E. Murch, Nashua, NH (US)

Correspondence Address: GUERIN & RODRIGUEZ, LLP, 5 MOUNT ROYAL AVENUE, MOUNT ROYAL OFFICE PARK, MARLBOROUGH, MA, 01752, US

(73) Assignee: Memtec Corporation, Salem, NH (US)

(21) Appl. No.: 10/942,198

(22) Filed: Sep. 16, 2004

(51) Int. Cl. A61B 5/00

(52) U.S. Cl. 600/301

(57) ABSTRACT

Described is a portable recorder for use by first-response emergency service personnel while providing care to a human subject. The recorder includes memory for storing data and sensor circuitry for sensing physiological signals through electrodes attached to a human subject and for converting the physiological signals into electrical signals. The recorder also has a transducer for converting sound waves into electrical signals and analog-to-digital converter circuitry for converting the electrical signals corresponding to the physiological signals and the electrical signals corresponding to the sound waves into digital data. A processing unit stores the digital data corresponding to the sound waves and the digital data corresponding to the physiological signals in the memory. The data stored in the memory are transferred to a remote computing system. An application program on that computing system automatically generates a report based on the data transferred from the memory.

### FIELD OF THE INVENTION

The invention relates generally to first-response portable recorders. More specifically, the invention relates to a system and a method for enabling emergency service providers to record information regarding an emergency event and to generate automatically a report based on the recorded information.

### BACKGROUND

Car accidents, heart attacks, gunshot wounds are but a few examples of life-threatening emergencies faced by members of society. Called to the scene of an emergency, trained emergency service personnel, such as paramedics and Emergency Medical Technicians or EMTs, are often the first to care for and treat a sick or injured person. A primary objective of an EMT is to administer emergency medical treatment, as needed, and to prepare the individual for transportation to a medical facility. Depending upon the circumstances, emergency service personnel may use special equipment, including devices that monitor vital signs, such as temperature, blood pressure, heart rate, and oxygen level in the person's blood. At the medical facility, the emergency service personnel may help transfer patients to the emergency department and report their actions to emergency room staff.

After the incident, the EMT or paramedic often needs to document the event. Information, such as the name, symptoms, and vital signs of the patient, the time and duration of the event, and the treatment administered, needs to become part of the report generated by the emergency service personnel. An EMT may need to recall some of this information from memory. However, in the exigency of the incident, the EMT may miss or fail to remember some important details. Alternatively, the EMT can manually record the information at the emergency scene; but generally this is undesirable because time spent recording details of the event detracts from the attention that the EMT can provide the emergency victim. On occasion, measurements of vital signs taken by the special equipment are to be included in the report. Integrating these measurements into the report take time and some measure of sophistication on behalf of the EMT. Disadvantageously, any time spent producing the report limits the EMT from responding to any new life-threatening situations. So as to be able to return quickly to an active status, an EMT may unintentionally rush the report and fail to include significant information. There is, therefore, a need for a system than helps the EMT capture the important information needed to document an incident and, subsequently, to simplify the generation of a report.

### SUMMARY

In one aspect, the invention features a portable recorder for use by first-response emergency service personnel while providing care to a human subject. The portable recorder includes persistent memory for storing data and sensor circuitry for sensing physiological signals through electrodes attached to a human subject and for converting the physiological signals into electrical signals. The recorder also includes a transducer for converting sound waves into electrical signals and analog-to-digital converter circuitry for converting the electrical signals corresponding to the physiological signals and the electrical signals corresponding to the sound waves into digital data. A processing unit stores the digital data corresponding to the sound waves and the digital data corresponding to the physiological signals in the persistent memory.

In another aspect, the invention features a method of recording an event. A portable recorder is provided. The portable recorder obtains physiological signals from a human subject and records sound including certain spoken keywords. The physiological signals and recorded sound are converted into digital data and stored in memory of the portable recorder.

In still another aspect, the invention features a method of generating a report. Data corresponding to digitally recorded sound and to physiological signals obtained from a human subject are retrieved from memory in which the data are stored. An application program is executed at a computing system that automatically generates a report based on the data retrieved from the memory.

In another aspect, the invention features an apparatus for use by first-response emergency service personnel while providing care to a human subject. The apparatus includes means for storing data; means for sensing physiological signals through electrodes that are attached to a human subject and for converting the physiological signals into electrical signals, means for converting sound waves into electrical signals; means for converting the electrical signals corresponding to the physiological signals and the electrical signals corresponding to the sound waves into digital data; and means for storing the digital data corresponding to the sound waves and the digital data corresponding to the physiological signals in the storing means.

In yet another aspect, the invention features a computer program product for use with a computer system. The computer program product includes a computer useable medium having embodied therein program code comprising program code for retrieving data corresponding to digitally recorded sound and to physiological signals obtained from a human subject from memory in which the data are stored, and program code for automatically generating a report based on the retrieved data corresponding to the digitally recorded sound and to the physiological signals.

### BRIEF DESCRIPTION OF THE DRAWINGS

The above and further advantages of this invention may be better understood by referring to the following description in conjunction with the accompanying drawings, in which like numerals indicate like structural elements and features in various figures. The drawings are not necessarily to scale, emphasis instead being placed upon illustrating the principles of the invention.

FIG. 1 is a view of an embodiment of a first-response data recording and report generating system of the present invention including a portable recorder and a computing system.

FIG. 2 is a detail sectional view of an embodiment of electrical leads of a communications port of the portable recorder of FIG. 1.

FIG. 3 is a diagram showing a human subject wearing a blood pressure cuff with the portable recorder of the invention being attached to the cuff.

FIG. 4 is a functional block diagram of an embodiment of the portable recorder of FIG. 1.

FIG. 5 is a functional block diagram of an embodiment of the computing system of FIG. 1.

FIG. 6 is a functional block diagram of an embodiment of a report generator program used in the system of FIG. 1 for automatically generating reports based on event data.

FIG. 7 is a flow diagram of an embodiment of a process for generating a report based on event data.

### DETAILED DESCRIPTION

In brief overview, the present invention features a portable recorder for use by emergency service providers, such as EMTs and firefighters, to record information regarding an emergency event and to generate a report based on the recorded information. The portable recorder may be referred to as a “first response” recorder because those emergency service personnel who arrive first on a scene to care for persons needing medical attention can carry the recorder in hand, in a bag, or from an article of clothing, such as a belt. Use of the recorder is not limited to emergency service personnel; other individuals can use the recorder in the performance of their duties, for example, home health care providers and insurance company representatives.

Combined in a single portable recorder are such capabilities as recording sound and measuring electrocardiogram signals, temperature, blood pressure, and the blood-oxygen content of the human subject. One embodiment of the portable recorder can also take and store digital photographs or video sequences. The various types of information gathered by the portable recorder are stored in a local memory. After being stored in the memory, the information is transferable to a remote computing system. A transfer can occur by wire or wireless transmission. Software executing on the remote computer processes the transferred information and automatically generates a document or report of the incident in accordance with a custom-designed report format.

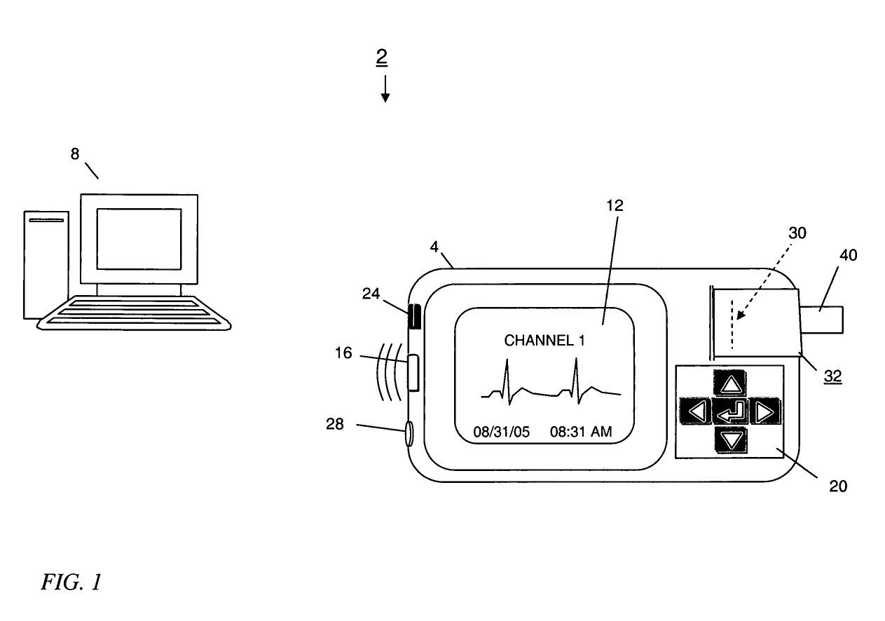

More specifically, FIG. 1 shows an embodiment of a first-response recorder and reporting system 2 including an embodiment of a first-response portable recorder 4 and a computing system 8. In one embodiment, the recorder 4 is generally rectangular, being approximately 5 inches in length, 3.5 inches wide, and 1.1 inches thick, and weighs approximately 8.7 ounces. One nine-volt alkaline battery, inserted into the recorder 4 from the backside, can operate the recorder 4.

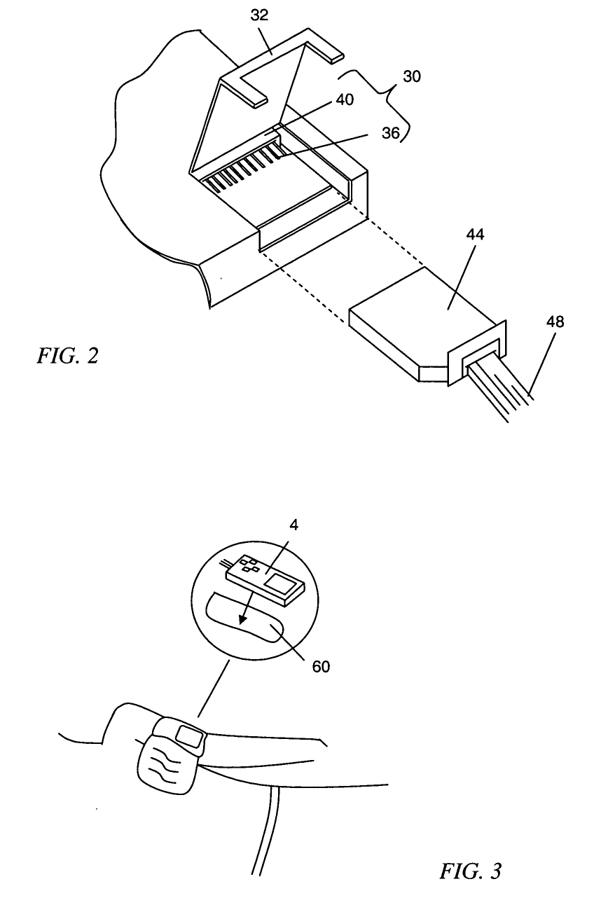

The recorder 4 includes a display screen 12, a wireless communications port 16, and a keypad 20 for navigating through dropdown menus to select and activate the various functional features of the recorder 4. For collecting various types of data, the recorder 4 includes means for recording sound, optional means 28 for taking digital photographs or for streaming video (i.e., a camera lens), and a wire communications port 30 hidden by a latched cover 32. FIG. 2 shows the cover 32 rotated into an open position, revealing a multi-lead 36 electrical connector 40 that mates with a cable 44 having a plurality of signal lines 48 (wires or electrodes). In one embodiment, the cable 44 includes a plurality of electrodes to collect physiological signals taken of a human subject. In other embodiments, the cable 44 is for performing USB or RS232 communications. In another embodiment, the cable 44 is used for digital photography. In this embodiment, one of the wires (having a lens at one end) carries an optical signal to an image sensor within the recorder 4 and to the display screen. The user holds the wire in one hand, points the lens at the end of the wire at the subject to be photographed, and looks at the display screen to see the image being captured.

In one embodiment, the means for recording sound includes a built-in microphone 24 (i.e., a transducer for converting sound waves into electrical signals). In another embodiment, the means for recording sound includes a wireless receiver in the recorder and a separate headset that the emergency service provider wears. The headset has a microphone and a wireless transmitter. With his or her hands free to assist a patient, the wearer of the headset can speak into the microphone. The headset's transmitter wirelessly transmits the spoken words to the receiver on the recorder, where the words are stored as audio data.

During operation, a first-response emergency service provider arriving on the scene attaches electrodes to the patient and turns on the recorder 4 to start making various forms of recording. Physiological measurements that may be taken by the recorder 4 include temperature, electrocardiogram or ECG signals, blood pressure, and pulse oximetry. Three or more electrodes 48 of the cable 44 are for obtaining the ECG signals, another electrode for taking the patient's temperature, and another electrode for the pulse oximetry. Patient vitals signs enter the recorder 4 through the wire communications port 30.

To measure the patient's blood pressure, the underside (not shown) of the recorder 4 can attach directly to the blood pressure cuff 60, as shown in FIG. 3. The cuff has a mechanical locking mechanism onto which the recorder snaps. The locking mechanism ensures an alignment of an O-ring on a backside of the recorder 4 with an opening on the blood pressure cuff. When the recorder 4 snaps on to the cuff, the O-ring forms a seal around the opening. A motor within the recorder 4 blows air into the cuff opening to inflate the cuff. The recorder controls this airflow and from the airflow determines the blood pressure information.

The physiological signals obtained by the electrodes may appear on the display screen 12 (e.g., a high-resolution dot matrix liquid crystal display). These vital signs may appear as a real-time waveforms (e.g., in the instance of ECG signals) or as computed values (e.g., systolic and diastolic blood pressure values). With the keyboard 20 the emergency service technician can select which vital signs to display. The keyboard 20 has four directional arrow keys for navigating through the menu items and a center key for selecting a highlighted item. The current date and time also appear in the display screen 12, along with the identity of the channel being visually monitored. The date and time automatically become part of the stored event data (e.g., when the user hits a button to store ECG data, data and time are stored with the ECG data. The date and time information differentiates data from different patients.

In addition to taking physiological measurements, other functional capabilities of the recorder 4 are to record sound and to take photographs and video. In one embodiment, the emergency service provider can selectively disable the sound recording. In another embodiment, the user cannot disable the recording of sound: the recorder 4 records sound by default when the user turns the recorder 4 on. In accordance with the invention, certain words spoken by the emergency service provider serve as keywords used by an application program (described below) to extract voice data that follow, to convert this voice data into text format, and to insert this text format into a particular location within a document. The emergency service provider can speak the keywords into the microphone of the recorder 4. To illustrate, while providing treatment the emergency service provider can say “Name: John Doe” into the mouthpiece of a headset that wirelessly transmits the spoken words to the microphone. Here, the keyword is “Name.” Other examples of keywords include, but are not limited to, a patient identification code, the patient's gender and age, and the time and date of the event. Keywords can be longer than one word.

In one embodiment, the recorder 4 automatically provides certain information, such as the patient identification code, so that the emergency service provider does not need to speak this information or the corresponding preceding keywords into the microphone.

In another embodiment, rather than say the keywords, the emergency service provider can enter keywords into the sound recording by pressing a certain button or buttons on the recorder 4. For example, a first press of the button can correspond to the keyword “patient's name” and a second press of the button can correspond to the keyword “gender.” After pressing the button once, the emergency service provider says the patient's name, and then after pressing the same button a second time, the emergency service provider says the patient's gender.

The recorder 4 can also take digital photographs and make streaming video recordings. To take a photograph or record a video, the user selects the appropriate menu item to activate the photograph or video capability and aims the camera lens 28 at the target subject. The subject matter being photographed or videoed appears in the display screen 12. To take the photograph or start the video, the user presses the center button of the keyboard 20. To stop a video, the center button is pressed a second time.

Obtaining physiological measurements or readings, recording sound, and acquiring photographic or video images produce various types of data, collectively referred to as event data, that the recorder 4 stores within local memory. Each type of data can be stored in a proprietary format or in a conventional format appropriate for that data type. For example, a conventional format for image data is JPEG and for audio data are MP3 and Wave. In one embodiment, the format used for the audio data is tailored for software used on the remote computing system 8 for converting that audio data into text.

After an event has ended, e.g., when the emergency service provider has transported a patient to a hospital, the event data can be transferred from the recorder 4 to the computing system 8, e.g., to a computer at the hospital or to a laptop computer within the emergency vehicle. This data transfer can occur wirelessly or over a wire connection, indirectly over a network or directly from the recorder 4 to the computing system 8. In one embodiment, the wireless transmission uses satellite-based communications. Infrared (IR) and radio frequency (RF) signals can be used in other embodiments to perform a wireless transmission. The use of encryption can secure the event data during the transfer.

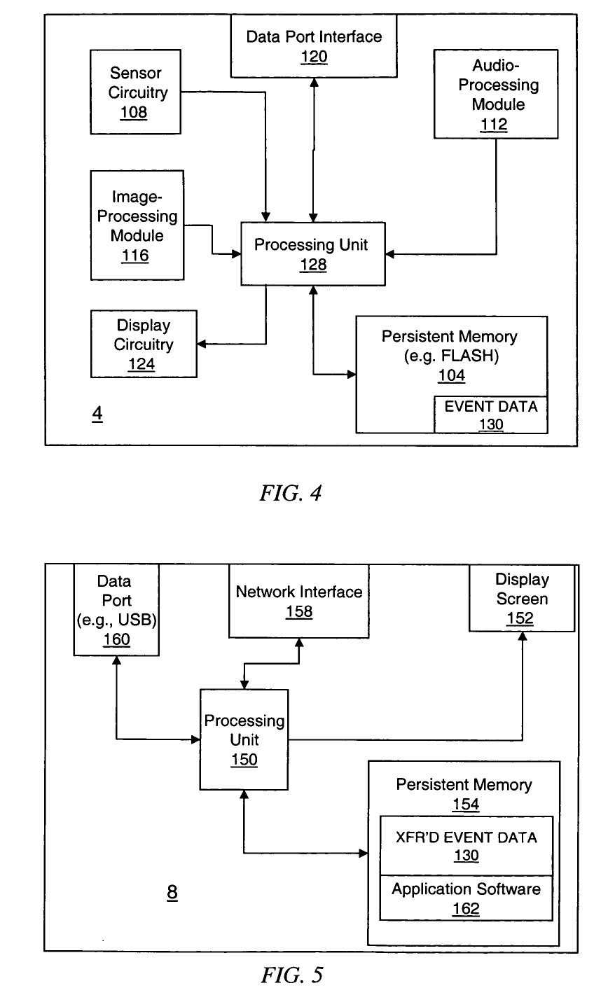

FIG. 4 shows a functional block diagram of an embodiment of the recorder 4 of the invention. The recorder 4 includes persistent memory 104, sensor circuitry 108, an audio processing module 112, an image-processing module 116, a data port interface 120, and display circuitry 124, each in electrical communication with a processing unit 128. In one embodiment, the persistent memory 104 is removable from the recorder 4 (such as the SmartMedia™ and compact FLASH memory cards commonly used in digital cameras). Embodiments of the memory 104 can range in size from 1 gigabyte to 5 gigabytes. The size of the memory 104 can store approximately 12 to 24 hours of data recordings (i.e., physiological data, audio data, and image data), although a large number of digital images and long video sequences can affect the number of hours for which free memory is available.

The sensor circuitry 108 is in communication with the electrodes 48 that attach to the human subject for obtaining the physiological readings. Sensor circuits acquire the various physiological signals from the human subject. Sensors detect electrical signals of the heart, the blood pressure of the subject, and the oxygen content of the blood. Signal conditioning circuitry (not shown) amplifies and filters the electrical signals to remove noise, and A-D converter circuitry changes the analog signals into digital (i.e., binary) data for storage in the memory 104. The sensor circuitry 108 can also include calculation circuitry for converting electrical signals into numerical values (e.g., the blood pressure) or for computing statistical values (e.g., average blood pressure).

The audio processing module 112 is in electrical communication with the means for recording sound to receive the electrical signals thereby produced, and to amplify, filter, and convert the electrical signals into digital data for storage in the memory 104. The image-processing module 116 includes an image sensor for converting the light that enters the camera lens 28 into electrical signals and for producing from these electrical signal digital data representing a detected image. Under the control of the processing unit 128, the digital data produced by the sensor circuitry 108, the audio data produced by audio processing module 112, and the image data produced by the image processing module 116 are stored in the memory 104. Also under the control of the processing unit 128, a copy of the event data can be transferred from the memory 104 to the external computing system 8 through the data port interface 120. The processing unit 128 also controls the content presented by the display circuitry 124 to the display screen 12.

Referring to FIG. 5, the computing system 8 includes a machine or device with a microcontroller or processor 150, a user interface, a display screen 152, an input device (e.g., keyboard, a mouse, trackball, touch-pad, touch-screen, etc), and persistent storage 154 for storing data and software programs. Exemplary embodiments of the computing system 8 include, but are not limited to, a personal computer, a Macintosh computer, a workstation, and a laptop computer. The operating system of the computing system 8 can be one of a variety of platforms including, but not limited to, WINDOWS 98, WINDOWS NT, WINDOWS XP, Macintosh, UNIX, and Linux. The computing system 8 can also be connected to a network, such as a local area network (LAN) or a wide area network (e.g., the Internet), through a network interface 158, and to other devices through a data port 160 (e.g., a USB port).

One application program stored in the persistent memory 154 of the computing system 8 is a report generator program 162 that automatically generates a document or report based on the event data 130. After the event data 130 are transferred to the computing system 8, a user can execute the report generator program 162 to produce a report. The user can save the report in the local memory 154 of the computing system 8, transfer a copy of the report to the recorder 4, print the report, or email the report. The user can also email a copy of the event data 130, if emailing is possible because of the size (in bytes of memory) of the event data. On the computing system 8, the emergency service provider can replay the voice recordings, view images, and analyze the monitored data. This can be done using the report generator program 162 or other software, conventional or proprietary, developed for such purposes.

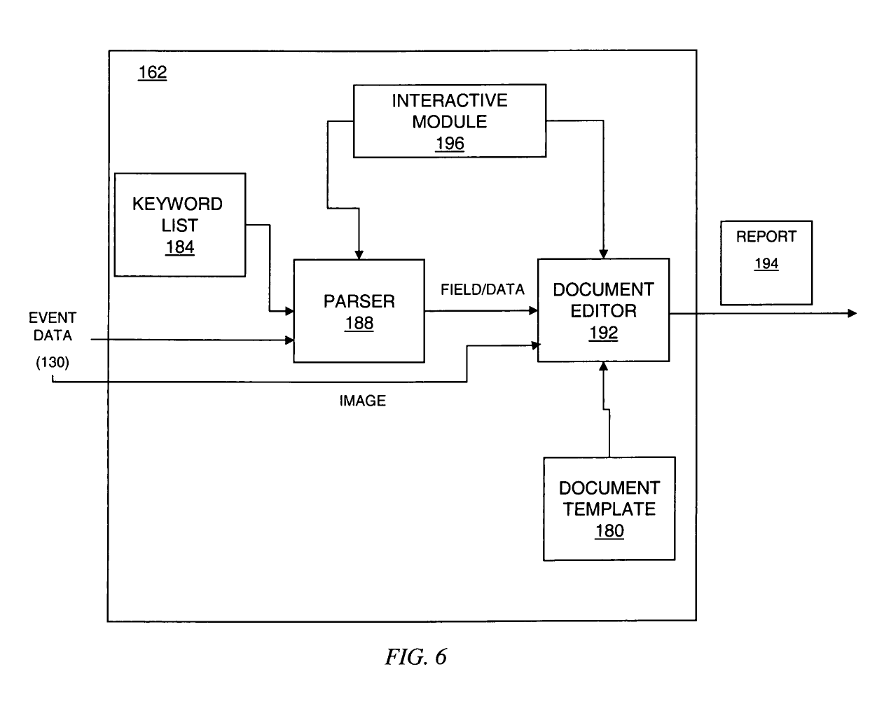

FIG. 6 illustrates an embodiment of the report generator program 162 of the invention. The report generator program 162 includes a document template 180, a keyword list 184, a parser 188, a document editor 192, and an interactive module 196. The document template 180 defines the format of the report to be generated, the content that appears in each document produced from this template, and those fields in the report that need to be filled in with information taken from the event data 130. The document template 180 may also identify the particular keywords to be included in the keyword list 184. In general, the document template 180 is customized according to the requirements of a particular organization or corporation (e.g., a hospital and insurance company).

The keyword list 184 defines those words that are used by the parser 188 to identify content to be extracted from the event data 130. Each keyword is associated with one or more particular fields in the document template 180. The parser 188 scans through the audio data of the event data 130 in search of keywords in the keyword list 184. In one embodiment, this audio data are preprocessed and converted into text data before the parser 188 searches for keywords. Alternatively, the parser 188 searches for keywords while converting the audio data into text. Processes for converting speech into text are generally known in the art.

Preferably, each keyword in the keyword list 184 is present in the event data 130 because the emergency service provider has recorded that keyword into the recorder 4 while attending to a patient (as described above). Upon detecting a keyword, in one embodiment the parser 188 extracts one or more words in the event data 130 that follow the detected keyword. Alternatively, the parser 188 can operate to extract one or more words that precede the detected keyword. The number of extracted words may be definable within the keyword list 184. Consider, for example, that the keyword “name” is associated with two words. When the parser 188 encounters the “name” keyword, the parser extracts the next two words in the audio data, presumably the first and last name of the patient. As another example, rather than counting words after a keyword the parser 188 can extract every word in the audio data between a “start” marker and a “stop” marker. That is, after speaking the keyword the emergency service provider says “Start,” then the information, and then “Stop.”

Each extracted word passes to the document editor 192, along with the identity of each field with which the extracted word is associated. The document editor 192 produces a new document or report 194 based on the document template 180 and fills in the appropriate fields with information provided by the parser 188. After the parsing of the audio data is complete, document editor 192 can launch interactive word processing software through which the user can correct any errors or misspelling in the automatically generated report 194. The generated report 194 can be text or multimedia and saved in a variety of conventional formats, e.g., as a WORD™ document, an HTML document, in rich text format, without departing from the principles of the invention.

The interactive module 196 communicates with the user of the report generator program 162 by prompting for input, receiving and processing that input. For example, the interactive module 196 can prompt the user to select an event data file and a document template upon which to base the report, or to browse through images in the event data 130 for inclusion in the report.

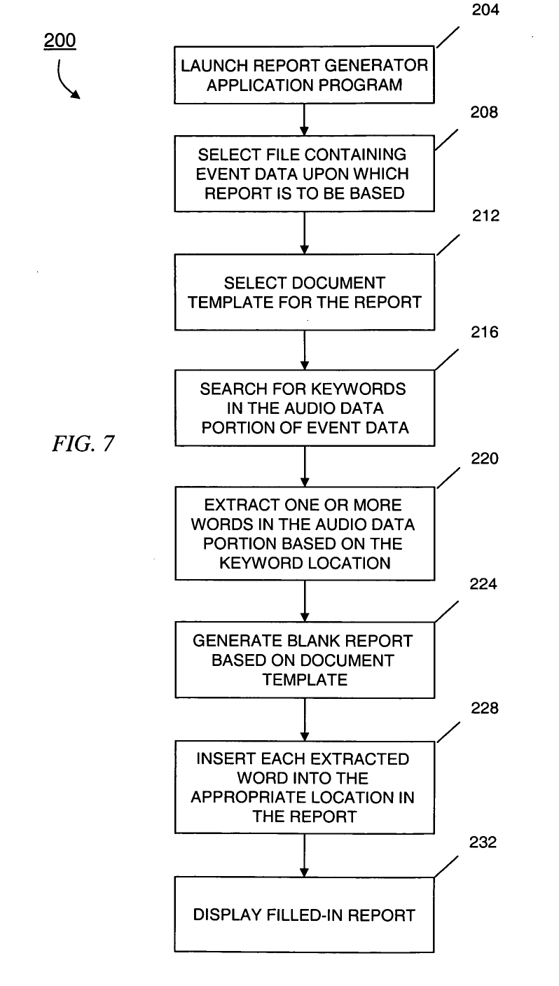

FIG. 7 shows an embodiment of a process 200 for automatically generating a report from event data. In the description of the process 200, reference is also made to FIG. 6. The user executes (step 204) the report generator program 162 and selects (step 208) a file containing the event data upon which the report is to be based. In one embodiment, the report generator program 162 selects (step 212) a default document template to use in constructing the report. Alternatively, the user can select a document template 180 to be used.

At step 216, the parser 188 parses through the audio data portion of the event data in search of keywords. Upon detecting a keyword, the parser 188 extracts (step 220) one or more words from the audio data. The document editor 192 generates (step 224) a blank report based on the document template 180 (i.e., having content, but with unfilled fields) and inserts (step 228) each extracted word into its associated field. When the parser 188 finishes parsing through the audio data portion, the document editor 192 can display (step 232) the filled-in report on the display screen of the computing system 8. The user then has opportunity to review and modify the automatically entered information.

If the event data includes an image, the interactive module 196 can ask the user whether the image should be included in the report. The interactive module 196 can also enable the user to browse through the available images in the event data and to select none, one or more of the images for inclusion in the report. Graphs of the physiological measurements and other statistics derived from the measured vitals signs, such as average blood pressure and oxygen content, can also be placed into the report automatically or under user direction. The report can also indicate the file name of the event data upon which the report is base and the name or identifier of the emergency service provider.

A portion of the present invention may be implemented as one or more computer-readable software programs embodied on or in one or more articles of manufacture. The article of manufacture can be, for example, any one or combination of a floppy disk, a hard disk, hard-disk drive, a CD-ROM, a DVD-ROM, a flash memory card, an EEPROM, an EPROM, a PROM, a RAM, a ROM, or a magnetic tape. In general, any standard or proprietary, programming or interpretive language can be used to produce the computer-readable software programs. Examples of such languages include C, C++, Pascal, JAVA, BASIC, Visual Basic, and Visual C++. The software programs may be stored on or in one or more articles of manufacture as source code, object code, interpretive code, or executable code.

Although the invention has been shown and described with reference to specific preferred embodiments, it should be understood by those skilled in the art that various changes in form and detail may be made therein without departing from the spirit and scope of the invention as defined by the following claims.

What is claimed is:

1. A portable recorder for use by first-response emergency service personnel while providing care to a human subject, the portable recorder comprising:
persistent memory for storing data;
sensor circuitry for sensing physiological signals through electrodes that are attached to a human subject and for converting the physiological signals into electrical signals;
a transducer for converting sound waves into electrical signals;
analog-to-digital converter circuitry for converting the electrical signals corresponding to the physiological signals and the electrical signals corresponding to the sound waves into digital data; and
a processing unit for storing the digital data corresponding to the sound waves and the digital data corresponding to the physiological signals in the persistent memory.

2. The portable recorder of claim 1, further comprising an image sensor for use in digitally recording an image.

3. The portable recorder of claim 1, wherein the physiological signals are at least one of ECG signals, blood pressure measurements, and oxygen content measurements.

4. The portable recorder of claim 1, further comprising a bottom surface having means for attaching to a blood pressure cuff.

5. The portable recorder of claim 1, further comprising electrodes for attaching to the human subject to obtain the physiological signals.

6. A method of recording an event, comprising:
providing a portable recorder;
obtaining by the portable recorder physiological signals from a human subject;
recording by the portable recorder sound including certain spoken keywords;
converting the physiological signals and recorded sound into digital data;
storing the digital data in memory of the portable recorder.

7. The method of claim 6, further comprising transmitting the digital data to memory of a remote computing system.

8. The method of claim 7, wherein the step of transmitting includes a wireless transmission of the digital data.

9. The method of claim 7, wherein the step of transmitting includes removing the memory from the portable recorder and electrically connecting the memory to a data port of the remote computing system.

10. The method of claim 6, further comprising sensing by the portable recorder a digital image and storing data corresponding to the digital image in the memory.

11. The method of claim 6, wherein the physiological signals are at least one of ECG signals, blood pressure measurements, and oxygen content measurements.

12. A method of generating a report, comprising:
retrieving data corresponding to digitally recorded sound and to physiological signals obtained from a human subject from memory in which the data are stored; and
automatically generating a report based on the retrieved data corresponding to the digitally recorded sound and to the physiological signals.

13. The method of claim 12, wherein the step of automatically generating the report includes detecting a keyword in audio data corresponding to the digitally recorded sound, converting one or more words that are near the keyword in the audio data into a text format, and inserting each text format word into a predetermined location within the report.

14. The method of claim 12, wherein the step of automatically generating the report includes inserting data corresponding to the physiological signals into a location in the report.

15. The method of claim 12, wherein the step of automatically generating the report includes producing a time-based graph of the data corresponding to the physiological signals and inserting the graph into a location in the report.

16. The method of claim 12, further comprising retrieving data corresponding to a digitally recorded image, and wherein the step of automatically generating the report includes inserting the image into a location in the report.

17. The method of claim 12, further comprising generating a template for the report, the template having one or more fields for receiving information based on audio data stored in the memory.

18. The method of claim 12, wherein the physiological signals are at least one of ECG signals, blood pressure measurements, and oxygen content measurements.

19. Apparatus for use by first-response emergency service personnel while providing care to a human subject, the apparatus comprising:
means for storing data;
means for sensing physiological signals through electrodes that are attached to a human subject and for converting the physiological signals into electrical signals;
means for converting sound waves into electrical signals;
means for converting the electrical signals corresponding to the physiological signals and the electrical signals corresponding to the sound waves into digital data; and
means for storing the digital data corresponding to the sound waves and the digital data corresponding to the physiological signals in the storing means.

20. The apparatus of claim 19, further comprising means for sensing light and converting the sensed light into digital data representing an image.

21. A computer program product for use with a computer system, the computer program product comprising a computer useable medium having embodied therein program code comprising:
program code for retrieving data corresponding to digitally recorded sound and to physiological signals obtained from a human subject from memory in which the data are stored; and
program code for automatically generating a report based on the retrieved data corresponding to the digitally recorded sound and to the physiological signals.

22. The computer program product of claim 21, wherein the program code for automatically generating the report includes program code for detecting a keyword in audio data corresponding to the digitally recorded sound, program code for converting one or more words that are near the keyword in the audio data into a text format, and program code for inserting each text format word into a location within the report.
